(19) United States
(12) Patent Application Publication
(10) Pub. No.: US 2001/0056086 A1
(43) Pub. Date: Dec. 27, 2001

(54) USE OF ANTI-OESTROGENS AS MALE CONTRACEPTIVES

(76) Inventor: URSULA-FRIEDERIKE HABENICHT, BERLIN (DE)

Correspondence Address: MILLEN WHITE ZELANO & BRANIGAN, ARLINGTON COURTHOUSE PLAZA I, 2200 CLARENDON BOULEVARD, SUITE 1400, ARLINGTON, VA, 22201

(21) Appl. No.: 08/913,304

(22) PCT Filed: Mar. 15, 1996
(86) PCT No.: PCT/EP96/01191

§ 371 Date: None

(30) Foreign Application Priority Data
Mar. 16, 1995 (DE) 195 10 862.0

(51) Int. Cl. A61K 31/56; A61K 31/47; A61K 31/41; A61K 31/135

(52) U.S. Cl. 514/177; 514/182; 514/381; 514/646

(57) ABSTRACT

This invention describes the use of antiestrogens (aromatase inhibitors, estrogen receptor antagonists) for the production of pharmaceutical agents for male birth control.

This invention relates to the use of antiestrogens for the production of pharmaceutical agents for male birth control.

In the more narrow sense, antiestrogens comprise the class of substances of compounds that can displace estrogens from their respective receptors (estrogen receptor antagonists) and, in the broader sense, also the compounds that prevent the synthesis of estrogens from their metabolic precursors in the organism—androgenic compounds with a 3-keto-4-ene steroid structure—by inhibiting the enzyme aromatase (aromatase inhibitors). These two groups, which in final analysis inhibit the biological action of estrogens, fall into the category of both steroidal and non-steroidal compounds in each case. In addition to the so-called pure antiestrogens, such as, e.g., 7α-[9-(4,4,5,5,5-pentafluoropentylsulfinyl)-nonyl]-estra-1,3,5(10)-triene-3,17β-diol, those compounds that, in addition to their antagonistic action, also have considerable agonistic, i.e., estrogenic, action, are among the competitive antiestrogens. The most prominent representative of the latter is tamoxifen.

There are already a considerable number of indications for which antiestrogens can be used. The best-known example is the clinical treatment of breast cancer with tamoxifen, which has been practiced for a long time.

The use of antiestrogens (centchroman) for female contraception in humans is also described (Nittyanand, S., Kamboj VP [1992] Centchroman: Contraceptive Efficacy and Safety Profile. International Conference on Fertility Regulation, November 5-8, 1992 Bombay, India, Programs and Abstracts). At effective dosages, however, undesirable side-effects such as, for example, osteoporotic changes, occur, which can be attributed to the systemic action of antiestrogens. Estrogen deprivation, which can occur after long-term treatment with an antiestrogen, limits at least their regular use for female contraception.

Finally, DE-A 42 13 005 describes the use of aromatase inhibitors for contraception in female primates of reproductive age at a dosage, in which the menstrual cycle of the female primate remains basically unaffected. In this case, the absolute level of the daily doses that are required for contraceptive action depends completely on the type of aromatase inhibitor that is used. For highly active aromatase inhibitors, the daily doses are generally between about 0.05 and about 30 mg. In the case of less active aromatase inhibitors, the daily doses can also be higher.

For male birth control, until now only condoms and vasectomy have been available. The former are only conditionally suitable both in terms of acceptance and in contraceptive reliability; vasectomies are generally irreversible with respect to fertility. A hormonal contraceptive that would be comparable to the oral contraceptives for women with respect to effectiveness, reliability, type of use, and acceptance was previously not in the offing. A further major advantage of hormonal contraception in women is its reversibility.

A summary of the current state of efforts for the development of a contraceptive for men is found in U. F. Habenicht in “Sitzungsberichte der Gesellschaft Naturforschender Freunde zu Berlin [Minutes of the Society of Friends of Natural Science in Berlin],” Volume 31, 1991, pp. 101-116.

Neither direct inhibition of spermatogenesis by various alkylating agents or the gossypols that have become known as “China pills” nor indirect inhibition of spermatogenesis by blocking the hypophyseal-hypothalamic system by using testosterone derivatives of LHRH analogues (agonists or antagonists) in combination with testosterone derivatives or with a combination of an androgen with a gestagen has yet produced the desired success.

In final analysis, the described attempts do not meet at least one of the two most important requirements of a modern contraceptive for men, namely the requirement for method reversibility and the lowest possible potential for side-effects.

In addition, the use of antigestagens (competitive progesterone receptor antagonists) for male birth control was also described (DE-A-40 39 561.8).

In treating male bonnet monkeys with RU 486 (11β-[(4-N,N-dimethylamino)-phenyl]-17β-hydroxy-17α-propinyl-4,9(10)-estradien-3-one, reduction of ejaculation weight, sperm count per ejaculation, reduction in sperm mobility, morphologic anomalities of sperm, and a loss/inhibition of acrosomes were observed.

The object of this invention is to provide a pharmaceutical agent for reversible control of male fertility which, in comparison to the already proposed pharmaceutical agents for this indication, is to exhibit fewer side-effects or better manageability.

This object is achieved by using antiestrogens for the production of pharmaceutical agents for male birth control.

It has now been found that antiestrogens, surprisingly enough, alter the acrosomal status of sperm: Thus, under the influence of antiestrogens, an incipient acrosomal reaction is observed. At the same time, the motility of the sperm is impaired by the antiestrogens.

The early induction of acrosomal reaction and the limitation of sperm motility may suggest that the latter are incapable of fertilization.

On the other hand, various parameters of the male sexual functions remain unaffected by antiestrogens: the organ weight of the male reproductive tract and the sperm concentration are not altered.

Thus, reversible inhibition of sperm functions, which are essential for successful fertilization, is produced by the antiestrogens.

These results are all the more astonishing as antiestrogens, such as, e.g., tamoxifen or clomiphene, have been used in certain male patients to correct fertility disorders [Acosta et al., Fertil. Steril. 55, pp. 1150-6, (1991)]. As a result, a locally increased estrogen concentration that is assumed to be present in the testes is to be counteracted, which possibly could be the cause of fertility disorders. In these patients, two active components of the antiestrogens used are at work: on the one hand, the antiestrogenic action per se, and on the other the endogenic testosterone increase due to the feedback mechanism (counterregulation).

The above-described properties of antiestrogens were obtained in tests that were performed with the ICI-antiestrogen 7α-[9-(4,4,5,5,5-pentafluoropentylsulfinyl)-nonyl]-estra-1,3,5(10)-triene-3,17β-diol on normal adult male rats.

This compound can be regarded as a standard compound for all compounds of this class of substances.

The test design and the results are described in the list below:

| | |
|---|---|
| Rats | normal male animals, weight |
| | about 200 g |
| group treated with ICI 7α-[9- | 2.5 mg/kg in a 0.2 ml vehicle |
| (4,4,5,5,5- | subcutaneously on an oily base |
| pentafluoropentylsulfinyl)- | (benzylbenzoate/castor oil) |
| nonyl]-estra-1,3,5(10)-triene- | |
| 3,17β-diol (n = 6; number of | |
| animals) | |
| Vehicle control (n = 6; number | 0.2 ml of vehicle 1 × daily |
| of animals) | |
| Period of treatment | 28 days |
| Determination of the organ | seminal vesicles, prostate, |
| weight of the male | testicles, epididymis |
| reproductive tract and | |
| histology | |
| Extraction of sperm | Extraction from the epididymis |
| | (epididymal sperm) |
| Determination of: | -- motility |
| | -- number |
| | -- acrosomal state |
| | (corresponding to the WHO-rich |
| | lines) |

Observations regarding the organ weight of the male reproductive tract and sperm properties:

1. No effects on the weight or histology of the organs studied

2. Inhibition of motility

3. Induction of an early incipient acrosomal reaction.

These observations clearly show that antiestrogens are suitable for the production of pharmaceutical agents for male birth control.

As compounds that have an antiestrogenic action, both competitive antiestrogens (estrogen receptor antagonists) and aromatase inhibitors according to the invention are suitable. Estrogen receptor antagonists and aromatase inhibitors according to this invention can be derived from both steroids or non-steroidal compounds. Compounds that have an antiestrogen action, in the broadest sense, are to be defined according to this invention only as those compounds that have the most selective action possible, i.e., that basically inhibit only the action of estrogens and/or reduce their concentration.

The estrogen receptor antagonists have a competitive action, by displacing estrogens from the receptor, while aromatase inhibitors inhibit the biosynthesis of estrogens. According to this invention, compounds of the aminoglutethimide type, i.e., 3-(4-aminophenyl)piperidine-2,6-diones that are alkylated in 3-position, etc., which in addition to the estrogen level also exert a reducing action on other sexual hormone serum concentrations, are not suitable as compounds that have an antiestrogenic action.

As non-steroidal estrogen receptor antagonists, there can be mentioned, for example:

Tamoxifen=(Z)-2-[p-(1,2-diphenyl-1-butenyl)-phenoxy]-N,N-dimethylethylamine,

nafoxidine=1-2-[4-(6-methoxy-2-phenyl-3,4-dihydro-1-naphthyl)-phenoxy]-ethylpyrrolidine, hydrochloride,

Mer 25=1-[p-(2-diethylaminoethoxy)-phenyl]-2-(p-methoxyphenyl)-1-phenylethanol

raloxifene=6-hydroxy-2-(p-hydroxyphenyl)benzo[b]thien-3-yl-p-(2-piperidino-ethoxy)phenyl ketone, hydrochloride;

centchromane

other compounds of 1,1,2-triphenylbut-1-ene type, especially 1,1-bis-(3′-acetoxyphenyl)-2-phenyl-but-1-ene [J. Cancer Res. Clin. Oncol., (1986), 112, pp. 119-124];

also suitable as steroidal estrogen receptor antagonists are, for example:

11α-methoxy-17α-ethinyl-1,3,5(10)-estratriene-3,17β-diol and 16β-ethylestradiol, N-n-butyl-N-methyl-11-(3,17β-dihydroxyestra-1,3,5(10)-trien-7α-yl)-undecanamide and

7α-[9-(4,4,5,5,5-pentafluoropentylsulfinyl)nonyl]estra-1,3,5(10)-triene-3,17β-diol.

As aromatase inhibitors, all compounds are suitable that are suitable as substrates for aromatase, such as, for example, the 1-methyl-androsta-1,4-diene-3,17-dione (atamestane) that is described in German Laid-Open Specification 33 22 285), the testolactone (17α-oxa-D-homoandrost-1,4-diene-3,17-dione) that is described in Journal of Clinical Endocrinology and Metabolism, 49, 672 (1979), the compounds that are described in “Endocrinology” 1973, Vol. 92, No. 3, page 874:

androsta-4,6-diene-3,17-dione,

androsta-4,6-dien-17β-ol-3-one-acetate,

androsta-1,4,6-triene-3,17-dione,

4-androstene-19-chloro-3,17-dione,

4-androstene-3,6,17-trione,

the 19-alkynylated steroids that are described in German Laid-Open Specification 31 24 780,

the 10-(1,2-propadienyl)-steroids that are described in German Laid-Open Specification 31 24 719,

the 19-thio-androstane derivatives that are described in European Patent Application, Publication No. 100 566,

the 4-androsten-4-ol-3,17-dione that is described in “Endocrinology” 1977, Vol. 100, No. 6, page 1684 and U.S. Pat. No. 4,235,893 and its esters,

the 1-methyl-15α-alkyl-androsta-1,4-diene-3,17-diones that are described in German Laid-Open Specification 35 39 244,

the 10β-alkinyl-4,9(11)-estradiene derivatives that are described in German laid-open specification 36 44 358 and the 1,2β-methylene-6-methylene-4-androstene-3,17-dione that is described in European Patent Application 0 250 262.

As non-steroidal aromatase inhibitors, for example, [4-(5,6,7,8-tetrahydroimidazo [1,5α]-pyridin-5-yl)benzonitrile-mono-hydrochloride] (Cancer Res., 48, pp. 834-838, 1988) and the cycloalkylenazoles that are described in EP-A-0 411 735 can be mentioned. The best-known representative of the last-mentioned compounds is the pentrozole that was already mentioned.

In addition, the compounds that were specifically mentioned as aromatase inhibitors in DE-A 42 13 005 can be used within the scope of this invention.

This list is not exhaustive; other antiestrogens that are described in the above-mentioned publications, as well as those from the publications that are not mentioned here, are also suitable.

The antiestrogens can be used according to this invention for suppressing male fertility according to different treatment schemes.

1. Intermittent treatment One-time daily to weekly oral treatment over 4-12 months. Then: a treatment-free interval of 3-5 months. After that, renewed treatment as above.

2. Continuous treatment One-time daily oral administration or oral administration at two-day to at most seven-day regular intervals or administration of depot formulations at regular intervals (e.g., 1× per month, 1× per quarter, etc.).

To produce a pharmaceutical agent for male birth control, the antiestrogens are used in a daily amount of 0.1 to 100 mg p.o. tamoxifen or an equivalent-action amount of another antiestrogen.

In the case where a depot formulation is used for the production of the pharmaceutical agent according to the invention, this depot formulation is selected in such a way that the daily rate of release of antiestrogen is 0.1 to 100 mg of tamoxifen or an equivalent-action amount of another antiestrogen.

Equivalent-action amounts of other antiestrogens, i.e., amounts that correspond to the indicated amount of tamoxifen for the inhibition of male fertility, can be determined, for example, in the uterus growth-inhibiting test after estrogen stimulation.

In the case of the production of oral dosage units, the formulation of antiestrogens for the purposes of this invention is done completely analogously to the already known use of tamoxifen (Eur. J. Cancer Clin. Oncol., 1985, 21, 985 and J. S. Patterson, “10 Years of Tamoxifen in Breast Cancer” in Hormonal Manipulation of Cancer; Peptides, Growth Factors and New (Anti)steroidal Agents, Raven Press, New York (1987)).

For the antiestrogen to be used in a depot formulation, it can be prepared as a microcrystal suspension, as an oily solution, or in the form of a vehicle that contains active ingredients (transdermal system).

The following examples are used to give a more detailed explanation of the invention.

### EXAMPLE 1

20.0 mg of tamoxifen (antiestrogen with agonistic partial action)

140.0 mg of lactose

55.0 mg of corn starch

2.5 mg of poly-N-vinylpyrrolidone 25

2.0 mg of aerosil

0.5 ma of magnesium stearate

220.0 mg total weight of the tablet, which is produced in the usual way on a tablet press. The active ingredient according to the invention optionally also can be pressed with respectively half the above-indicated additives separately into a two-layer tablet.

### EXAMPLE 2

5.0 mg of 7β-[9-(4,4,5,5,5-Pentafluoropentylsulfinyl)-nonyl]estra-1,3,5(10)-triene-3,17β-diol (pure antiestrogen)

150.0 mg of lactose

60.0 mg of corn starch

2.5 mg of poly-N-vinylpyrrolidone 25

2.0 mg of aerosil

0.5 mg of magnesium stearate

220.0 mg total weight of the tablet, which is produced in the usual way on a tablet press. The active ingredient according to the invention optionally also can be pressed with respectively half the above-indicated additives separately into a two-layer tablet.

### EXAMPLE 3

0.2 mg of 5-[Cyclopentylidene-(1-imidazolyl)-methyl]-thiophene-2-carbonitrile (aromatase inhibitor-pentrozole)

160.0 mg of lactose

54.8 mg of corn starch

2.5 mg of poly-N-vinylpyrrolidone 25

2.0 mg of aerosil

0.5 mg of magnesium stearate

220.0 mg total weight of the tablet, which is produced in the usual way on a tablet press. The active ingredient according to the invention optionally also can be pressed with respectively half the above-indicated additives separately into a two-layer tablet.

1. Use of antiestrogens for the production of pharmaceutical agents for male birth control.

2. Use of estrogen receptor antagonists according to claim 1.

3. Use of aromatase inhibitors according to claim 1.

4. Use of tamoxifen according to claim 2.

5. Use of 7β-[9-(4,4,5,5,5-pentafluoropentylsulfinyl)nonyl]estra-1,3,5(10)-triene-3,17β-diol according to claim 2.

6. Use of atamestane according to claim 3.

7. Use of pentrozole according to claim 3.
